(19) United States
(12) Patent Application Publication
(10) Pub. No.: US 2006/0216302 A1
(43) Pub. Date: Sep. 28, 2006

(54) Immunological markers

(76) Inventor: Robert Root-Bernstein, East Lansing, MI (US)

Correspondence Address: HARNESS, DICKEY & PIERCE, P.L.C., P.O. BOX 828, BLOOMFIELD HILLS, MI, 48303, US

(21) Appl. No.: 10/547,046

(22) PCT Filed: Feb. 27, 2004
(86) PCT No.: PCT/US04/06030

§ 371 Date: Aug. 26, 2005

(30) Foreign Application Priority Data
Feb. 28, 2003 (US) 60451194

(51) Int. Cl. C12Q 1/70; G01N 33/554; A61K 39/00

(52) U.S. Cl. 424/185.1; 514/13; 514/14; 514/12; 435/5; 435/7.32

(57) ABSTRACT

Compounds, compositions and methods for identifying human or other animals having a particular characteristic, by administration of an antigenic marker that elicits a unique antigenic response. Compositions comprise: (a) an antigenic marker; and (b) a pharmaceutically-acceptable carrier wherein said marker does not elicit a vaccination immune response. In a preferred embodiment, the composition additionally comprises a vaccination antigen effective against organisms such as Mycobacterium, human immunodeficiency virus (HIV), 10 hepatitis C, Espstein-Barr virus, cytomegalovirus virus, E. coli feline leukemia virus, foot-and-mouth virus, and canine distemper virus. The present invention also provides methods for tracking a subject having a given characteristic in a population of animal subjects. The tracking is performed by administering an antigenic marker to the individual to produce antibodies that are unique to the antigenic marker and then screening the population to identify the individual based on the presence of the marker antibodies.

### BACKGROUND

The present invention relates to immunological materials, compositions, and methods for identifying and tracking human or other animal subjects having certain characteristics, including individuals that have been vaccinated against infectious diseases. In particular, such methods include the identification of individuals who have been vaccinated so as to differentiate such individuals from individuals who have actually been infected.

Humans and other animals are protected against infectious diseases by various physical and biochemical means. Among those means is the immune system, which comprises a complex of highly specialized cells and biochemical agents that seek out, identify, and eliminate pathogens. Infection with a pathogen causes an immune response in the infected host. In most cases, the immune response increases in strength over time, until the pathogens are eliminated and the host recovers. However, in some instances, the host's immune response is insufficient, and serious illness or death may result.

A host's immune response to a pathogen is stimulated by proteins or other chemicals (antigens) that are substituents of the pathogen and foreign to the host. B cell lymphocytes, in particular, produce antibodies that circulate in the blood and lymph. These antibodies are highly specific to a given antigen. Some antibodies coat the pathogen, marking it for destruction by phagocytes. In other cases, the combination of the antibody with its antigen activates complement in the blood, which destroys the pathogen. In yet other cases, the antibody blocks viruses from entering cells. Following the elimination of the infecting pathogen, the quantity of antibodies produced by the immune system subsides. However, memory cell lymphocytes remain, with the ability to identify the pathogen and produce new antibodies if re-infection occurs.

Vaccines have been used for many years to protect human and other animal subjects against a variety of infectious diseases. Vaccines bring about immunity by provoking an immune response from the subject, activating lymphocytes and creating a memory in the immune system. The immune system is thus primed, so that it can quickly respond to exposure to the active disease. Conventional vaccines consist of attenuated pathogens (for example, polio virus), killed pathogens (for example, Bordetella pertussis) or immunogenic components of the pathogen (for example, diphtheria toxoid).

A wide variety of vaccines have been developed and are approved for human use, including those effective against anthrax, cholera, diphtheria, hepatitis A, hepatitis B, measles, pertussis, polio, rabies, rubella, smallpox, tetanus, and typhoid. Many others are under development, including those for human immunodeficiency virus (HIV). One of the issues presented by the use of vaccines, however, is the inability to differentiate between subjects who have been immunized, and those who are actually infected with the disease. Such differentiation is highly important for screening of blood for potential pathogens and for public health surveillance. The issue is somewhat less significant for acute disorders, where the infection is readily ascertained by its symptoms and resolved leaving the blood of the subject safe for transfusion. For example, in screening women for exposure to Rubella (“German measles”), it is irrelevant whether immunity (if present) was originally induced by natural infection or vaccination. In both cases, the mother and her fetus are protected.

However, the need to distinguish individuals who are vaccinated from those infected is particularly keen for chronic and latent infections, where symptoms may not be readily ascertained by means other than immunological tests (antibody detection), and where infection can be transmitted through the blood. One such example is tuberculosis (TB). The primary screen for TB is a delayed-type hypersensitivity (DTH) to purified protein derivative (PPD) introduced subcutaneously. The screen cannot differentiate between natural and artificial immunity. Thus, despite the existence of an effective vaccine (Bacillus Callmette-Guerin, or BCG) against TB that is widely used in Scandinavian and other countries, public health authorities in the United States and some other countries have argued against the use of BCG on the grounds that its use would make it impossible to screen for or to track the incidence of new cases of TB. Thus, everyone in the latter countries is left susceptible to TB in order that new cases be identifiable and treated.

Another problem is posed by the development of HIV vaccines. Currently, all blood donations and organ transplants are screened for the presence of antibodies to HIV, and are discarded if HIV antibodies are present. This procedure will no longer be acceptable once HIV vaccination begins, since it will result in the discarding of ever-larger proportions of donated blood and organs. Similar problems are posed by vaccination for latent and chronic viruses such as the hepatitis virus (types A, B and C), cytomegalovirus (CMV) and Epstein-Barr virus (EBV). Public health officials are faced with a difficult choice between allowing vaccination in order to prevent infection, or banning vaccination so as to retain the benefit of being able to identify, track, and treat endemic disease.

The prior art attempts to distinguish between vaccinated and infected animals have been limited. Modification of the syringe used to vaccinate animals has been proposed, so that upon injection, the vaccinated subject would be marked with ink on the skin. See, U.S. Pat. No. 6,264,637, Hogan, issued Jul. 24, 2001. Such methods are inapplicable to vaccines administered by intraperitoneal, intragastric, intranasal, oral, genital or ocular routes. Additionally, using ink to mark an animal that has been vaccinated would require that an extremely large range of colors be used, resulting in difficult and burdensome decoding.

Another approach suggested in the art is to purify the wild type vaccine to contain only the proteins essential for protection. Non-essential proteins (NSP; non-structural proteins) are excised during the purification process. Vaccinated subjects would, accordingly, lack antibodies against the NSPs, unlike infected subjects. See, e.g., U.S. Pat. No. 6,013,266, Segers et al., issued Jan. 11, 2000. However, altering the wild type vaccine may change the effectiveness of the vaccine, limiting the scope of animals that can produce the corresponding antibodies, or cause undesired side effects.

### SUMMARY

The present invention provides compounds, compositions and methods for identifying human or other animal subjects having a particular characteristic, by administration to the subject of antigenic marker that elicits a unique antigenic response in the subject. Antigenic marker compositions of the present invention comprise:

(a) an antigenic marker; and

(b) a pharmaceutically-acceptable carrier;

wherein said marker does not elicit a vaccination immune response in said subject. In a preferred embodiment, the antigenic marker is a peptide or a peptoid. The present invention also provides antigenic markers, and methods of selecting antigenic markers, comprising:

(a) identifying a candidate peptide or peptoid marker having from about 8 to about 100 amino acid residues;

(b) determining the attribute score for each of said residues in said candidate marker, said attribute score being the average of the rarity, antigenicity and substitutability scores, on a scale of from 1 to 20, for each of said residues;

(c) calculating the average attribute score for all of said residues in said candidate marker; and

(d) selecting said candidate marker if said average attribute score is less than about 11.

In a preferred embodiment, the composition additionally comprises a vaccination antigen. The antigenic marker raises an immune response that is unique from the response of the vaccination antigen and other vaccination antigens. In a preferred embodiment, the compositions are effective vaccines against organisms selected from the group consisting of Mycobacterium, human immunodeficiency virus (HIV), feline leukemia virus, and foot-and-mouth virus.

In another embodiment, the marker composition does not additionally comprise a vaccination antigen. In a preferred embodiment, biomarker compositions are used to identify animals such as agricultural livestock and domestic companion animals. The present invention also provides methods for tracking a subject having a given characteristic in a population of animal subjects. The tracking is performed by administering an antigenic marker to the individual to produce antibodies that are unique to the antigenic marker and then screening the population to identify the individual based on the presence of the marker antibodies. In one embodiment, the animal subjects are administered a plurality of, preferably from 2 to 10, antigenic markers. Identification kits are also provided, for use in determining which subjects have been administered an antigenic marker. The present invention also provides a database for use in tracking individual subjects.

It has been found that the compounds, compositions and methods of this invention afford benefits over methods for identifying subjects among those known in the art. In particular, such benefits include one or more of being useful with vaccination antigens against a variety of diseases, not requiring modification of the vaccination antigen, being capable of administration through a variety of routes, increased specificity, enhanced reliability, enhanced ease of use, ease of development, and reduced cost. Other advantages will be apparent to one of ordinary skill in the art. It should be understood, however, that the detailed description and specific examples, while indicating embodiments among those preferred, are intended for purposes of illustration only and are not intended to limited the scope of the invention.

### DETAILED DESCRIPTION

The following definitions and non-limiting guidelines must be considered in reviewing the description of this invention set forth herein.

The headings (such as “Introduction” and “Summary,”) and sub-headings (such as “Markers”) used herein are intended only for general organization of topics within the disclosure of the invention, and are not intended to limit the disclosure of the invention or any aspect thereof. In particular, subject matter disclosed in the “Introduction” may include aspects of technology within the scope of the invention, and may not constitute a recitation of prior art. Subject matter disclosed in the “Summary” is not an exhaustive or complete disclosure of the entire scope of the invention or any embodiments thereof.

The citation of references herein does not constitute an admission that those references are prior art or have any relevance to the patentability of the invention disclosed herein. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the invention, are intended for purposes of illustration only and are not intended to limit the scope of the invention. Moreover, recitation of multiple embodiments having stated features is not intended to exclude other embodiments having additional features, or other embodiments incorporating different combinations of the stated features.

As used herein, the words “preferred” and “preferably” refer to embodiments of the invention that afford certain benefits, under certain circumstances.

However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the word “include,” and its variants, is intended to be non-limiting, such that recitation of items in a list is not to the exclusion of other like items that may also be useful in the materials, compositions, devices, and methods of this invention.

The present invention encompasses certain novel compounds, compositions and methods for the administration of antigenic marker compounds to human or other animal subjects. Specific compounds and compositions to be used in the invention must, accordingly, be pharmaceutically acceptable. As used herein, such a “pharmaceutically acceptable” component is one that is suitable for use with the intended human and/or other animal subject without undue adverse side effects (such as toxicity, irritation, and allergic response) commensurate with a reasonable benefit/risk ratio.

Markers:

The compositions and methods of this invention employ an “antigenic marker,” which as referred to herein, is any molecule which elicits an immune response involving the production of antibodies in a human or other animal subject when administered according to a method of this invention. Such antibodies include whole immunoglobulin (IgG) of any class, e.g., IgG, IgM, IgA, IgD, IgE, chimeric antibodies or hybrid antibodies with dual or multiple antigen or epitope specificities.

The marker is immunogenic, activating immune cells in the intended human or other animal subject to generate an immune response against the marker. Preferably the antigenic marker does not elicit a vaccination immune response in the subject. As referred to herein, a marker that “does not elicit a vaccination immune response” is an antigenic marker which is not a substituent of a vaccination antigen, and does not produce antibodies in a human or other animal subject to which it is administered that are substantially similar to the antibodies produced by the subject in response to a wild-type pathogen. Preferably, the antigenic markers are synthetic compounds that do not occur naturally, in particular not occurring in foods, medications, or other materials to which the animals or human subjects to which the marker is administered are likely to be exposed.

Preferably, the antigenic markers are antigenically unique, relative to antibodies produced by the subject to which they are administered in response to any other naturally-occurring antigen. Preferably the antigenic markers are also antigenically unique relative to other markers of this invention. As referred to herein, “antigenically unique” means that the subject antigenic marker is no more than about 50%, preferably no more than about 40%, more preferably no more than about 30% homologous with any naturally-occurring antigen or other antigenic marker with which the subject antigenic marker is being compared. Homology may be determined by use of any conventional alignment, similarity or homology algorithm. Computer programs executing such algorithms include BLAST, FASTA, and ALIGN, available through the ExPASy (Expert Protein Analysis System) Molecular Biology Server, provided by Geneva Bioinformatics (GeneBio) SA, Geneva Switzerland (www.expasy.com. Computer algorithms also include the GCG Wisconsin Package, marketed by Accelrys, Inc. (formerly Genetic Computer Group), San Diego, Calif., U.S.A. Also preferably, the marker does not create a significant adverse immune response, such as allergic response or immunosuppression, in the human or other animal subject to which it is administered.

Preferably, the marker is a peptide or peptoid, comprising from about from about 8 to 100 residues, preferably from about 8 to 40 residues, preferably from about 12 to 30 residues, more preferably from about 16 to 24 residues. As referred to herein, a “residue” is a monomer component of a peptide or peptoid polymer. As referred to herein, a “peptide” is a polymer comprising naturally occurring amino acids with naturally occurring linkages. As referred to herein, a “peptoid” is a polymer comprising naturally occurring amino acids with non-naturally occurring linkages, non-naturally occurring amino acid variants, antigenic sugar residues, or combinations thereof. Such amino acid variants include: those in which free amino groups have been derivatized to form amine hydrochlorides, p-toluene sulfonyl groups, carbobenzoxy groups, t-butyloxycarbonyl groups, chloroacetyl groups, or formyl groups; those in which free carboxyl groups have been derivatized to form salts, methyl and ethyl esters; those in which free hydroxyl groups have been derivatized to form O-acyl or O-alkyl groups; those in which imadazole groups (in histidine) are derivatized to form N-imbenzylhistidine; β-amino acids; α-amino acids with derivatized or otherwise non-naturally occurring side chains; D-amino acids; modified amino acids such as iodinated tyrosine, glycosylated glutamine and glycosylated lysine; phosphopeptides; glycopeptides; nucleopeptides; and chromopeptides. Such peptoids are among those described in The Peptides, Vol. 1, E. Schroeder et al., eds., 294-311. In one embodiment, the marker comprises a peptide consisting essentially of the common amino acids (i.e., the twenty amino acids that are commonly found in living organisms).

The markers of the present invention can be linked or conjugated to other chemical structures. In one embodiment, the antigenic marker comprises a peptide or peptoid having from 8 to 16 residues conjugated to a carrier peptide. Such carrier peptides includes those known in the art, such as keyhole limpet hemocyanin (KLH) and bovine serum albumen (BSA).

The antigenic markers may be neutral or in salt form. Pharmaceutically acceptable salts include the acid addition salts, formed with free amino groups of the peptide and inorganic acids such as, for example, hydrochloric or phosphoric acids, or organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

In an embodiment where the marker is a peptide, the marker preferably comprises at least one amino acid selected from the group consisting of His (histidine), Trp (tryptophan), Arg (arginine), Met (methionine), Gln (glycine), Tyr (tyrosine), Cys (cystine), Phe (phenylalanine), Asn (asparagine), Asp (aspartic acid), Lys (lysine), and mixtures thereof. More preferably, the marker comprises at least one amino acid selected from the group consisting of His, Trp, Arg, Met, Gln, Tyr, and mixtures thereof. Preferably the marker comprises at least abut 50%, more preferably at least about 60%, of such preferred amino acids. Also preferably, the markers do not comprise amino acids selected from the group consisting of Gly (glycine), Glu (glutamic acid), Ser (serine), Leu (leucine), Ile (isoleucine), and Thr (threonine), more preferably the group consisting of Gly, Glu, Ser, Leu, Ile, Thr, Lys (lysine), Val (valine), Pro (proline), Ala (alanine), and Asp (aspartic acid). Preferably the marker comprises no more than 50%, preferably no more than about 30%, preferably no more than about 15%, of such amino acids.

Preferably, the marker does not contain amino acid pairs and triplets that have a high-frequency of occurrence in natural proteins. Preferably, the marker comprises low-frequency and underrepresented pairs and triplets so as to minimize the probability that any particular sequence will mimic a naturally occurring peptide or protein sequence, and so as to minimize the possibility of cross-reactivity. In a preferred embodiment, the marker comprises the triplet Cys-His-Trp, and peptide pairs such as Trp-Pro, Met-Cys, and Cys-Glu.

Methods for Selecting a Marker:

The present invention provides methods for selecting a marker. As referred to herein, “selecting” is the determination of the chemical composition of a marker suitable for use in the compositions and methods of this invention. In a preferred embodiment, there are two basic criteria for selecting a marker. First, the candidate marker preferably neither cross reacts with, nor mimics antibody responses to, any pathogenic antigen. Second, the marker is preferably antigenically unique from every other marker of this invention that has been selected, produced and used in practice prior to the candidate marker. Among such markers useful herein are synthetic organic compounds having low-toxicity and high antigenicity, very unusual peptide sequences that do not occur naturally in living organisms, and mucopeptides bearing unnatural, antigenic sugar residues.

A preferred marker of the present invention comprises one or more sequences found to have a low probability of occurring naturally and which sequences contain the most antigenic amino acid residues. The amino acid residues used in a peptide marker are preferably those residues least capable of being substituted by another amino acid in the peptide sequence without loss of antigenicity. In this way, a peptide vaccination marker has the lowest possible immunologic cross-reactivity with other peptides of similar (but not identical) constitution, and therefore the lowest probability of yielding an unwanted immunologic reaction or a false vaccination marker test result.

The present invention provides methods of selecting a marker using measures of antigenicity, rarity and substitutability for each peptide or peptide residue in a candidate marker. As referred to herein, a “candidate” marker is a peptide or peptoid which has been isolated, synthesized, or identified for potential synthesis for consideration of use as an antigenic marker in a method of this invention. As referred to herein, “antigenicity” is defined as the relative occurrence of a particular amino acid residue within a known antigenic region as compared with the occurrence of that same residue in proteins in general. See, e.g., Geysen, et al. “Amino acid composition of antigenic determinants: Implication for antigen processing by the immune system of animals,” In: Lerner, et al, eds., Vaccines 85, NY: Cold Spring Harbor Laboratory, p. 133 (1985); and Welling, et al., “Prediction of sequential antigenic regions of proteins,” FEBS Lett 188: 215-219 (1985). As referred to herein, “rarity” is defined as the relative frequency with which a given amino acid residue sequence occurs in a naturally occurring peptide or protein. See, e.g., Welling et al., supra; and White, “Amino acid preferences of small proteins. Implications for protein stability and evolution,” J. Mol. Biol. 227: 991-995 (1992). As referred to herein, “substitutability” is defined as the relative degree of antigenicity loss occurring as a result of replacing or substituting a particular amino acid residue with another residue within an antigenic domain. See, e.g., Geysen, et al., “Cognitive features of continuous antigenic determinants,” In: J P Tam and E T Kaiser, eds., Synthetic Peptides. Approaches to Biological Problems, NY: Liss, pp. 19-30 (1989); and Gonzales, et al., “Comparison of capsid protein VP1 of the viruses used for the production and challenge of foot-and-mouth diseases vaccines in Spain,” Vaccine 10: 731-734 (1992). The following table sets forth the antigenicity, rarity, and substitutability scores, on a scale of 1-20, for the common amino acids.

TABLE 1
Attribute Scores

| | Anti- | | Substi- | Attribute Score |
|---|---|---|---|---|
| Amino Acid | genicity | Rarity | tutability | (average) |
| Ala (alanine) | 3 | 19 | 11 | 11 |
| Arg (arginine) | 6 | 12 | 3 | 7 |
| Asn (asparagine) | 8 | 8 | 8 | 8 |
| Asp (aspartic acid) | 5 | 11 | 15 | 10.3 |
| Cys (cystine) | 16 | 2 | 7 | 8.3 |
| Gln (glutamine) | 14 | 6 | 9 | 9.7 |
| Glu (glutamic acid) | 13 | 15 | 19 | 15.7 |
| Gly (glycine) | 18 | 18 | 14 | 16.7 |
| His (histidine) | 1 | 3 | 5 | 3 |
| Ile (isoleucine) | 19 | 13 | 10 | 14 |
| Leu (leucine) | 4 | 20 | 20 | 14.7 |
| Lys (lysine) | 2 | 10 | 18 | 10 |
| Met (methionine) | 20 | 4 | 2 | 7 |
| Phe (phenylalanine) | 17 | 7 | 6 | 10 |
| Pro (proline) | 12 | 9 | 13 | 11.3 |
| Ser (serine) | 10 | 17 | 16 | 14.3 |
| Thr (threonine) | 11 | 14 | 17 | 14 |
| Trp (tryptophan) | 15 | 1 | 1 | 5.7 |
| Tyr (tyrosine) | 7 | 5 | 4 | 5.3 |
| Val (valine) | 9 | 16 | 12 | 12.3 |

Rank ordering the Attribute Score for each amino acid in Table 1 yields an Aggregate Attribute Score of rarity, antigenicity and substitutability, set forth in the following Table 2. The lower the number, the more likely an amino acid is to be useful in designing a vaccination marker because it is less likely to occur in any protein, has a high relative antigenicity, and has high antigenic specificity (i.e., low substitutability).

TABLE 2
Aggregate Score

| Amino Acid | Aggregate Attribute Score |
|---|---|
| His (histidine) | 1 |
| Tyr (tyrosine) | 2 |
| Trp (tryptophan) | 3 |
| Met (methionine) | 4 |
| Arg (arginine) | 5 |
| Asn (asparagine) | 6 |
| Cys (cystine) | 7 |
| Gln (glutamine) | 8 |
| Phe (phenylalanine) | 9 |
| Lys (lysine) | 10 |
| Asp (aspartic acid) | 11 |
| Ala (alanine) | 12 |
| Pro (proline) | 13 |
| Val (valine) | 14 |
| Thr (threonine) | 15 |
| Ile (isoleucine) | 16 |
| Ser (serine) | 17 |
| Leu (leucine) | 18 |
| Glu (glutamic acid) | 19 |
| Gly (glycine) | 20 |

A preferred method of selecting a peptide marker comprises:

(a) identifying a candidate marker having from about 8 to about 100 amino acid residues;

(b) determining the attribute score for each of said residues in said candidate marker, said attribute score being the average of the rarity, antigenicity and substitutability scores, on a scale of from 1 to 20, for each of said residues;

(c) calculating the average attribute score for all of said residues in said candidate marker; and

(d) selecting said candidate marker if said average attribute score is less than about 11.

The rarity, antigenicity and substitutability scores are as set forth in Table 1, above. Preferably, the average attribute score is less than about 9, more preferably less than about 7. Also, preferably, the attribute score for each amino acid residue in the marker is less than about 11, more preferably less than about 10, more preferably less than about 8.5, and even more preferably less than about 7. Preferably, the marker comprises amino acids at least 50%, more preferably at least about 60%, more preferably at least about 70%, of which have an attribute score less than about 10, more preferably less than about 7.

In another embodiment, the present invention provides a method of selecting a marker comprising:

(a) identifying a candidate marker having from about 9 to about 100 amino acid residues;

(b) determining the aggregate score for each of said residues in said candidate marker, said aggregate score being the rank, from 1 to 20, of the rarity, antigenicity and substitutability scores for each of said residues;

(c) calculating the average aggregate score for all of said residues in said candidate marker; and

(d) selecting said candidate marker if said average aggregate score is less than about 12.

The aggregate score for each amino acid is as set forth in Table 2, above. Preferably, the average aggregate score is less than about 10, more preferably less than about 6, more preferably less than about 5. Also, preferably, the aggregate score for each amino acid residue in the marker is less than 11, more preferably less than about 10, more preferably less than about 7, and even more preferably less than about 5. Preferably, the marker comprises at least 50%, more preferably at least about 60%, more preferably at least about 70%, of amino acids which have an aggregate score less than about 10, more preferably less than about 7.

In another embodiment, the present invention provides a method of selecting a marker, comprising generating a peptide (herein, an “antisense peptide”) from the non-coding strand of a gene or gene fragment. Such antisense peptides may be generated by normal or backward transcription of the non-coding (c-DNA or complementary deoxyribonucleic acid) of a gene. Such genes useful herein include the genes from any organism; in one embodiment, the genes are from species of the subject to whom the marker is to be administered. Methods of identifying and making antisense peptides among those useful herein are disclosed in the following references: U.S. Pat. No. 5,077,195, Blalock et al., issued Dec. 31, 1991; U.S. Pat. No. 5,523,208, Kohler, et al., issued Jun. 4, 1996; and R. S. Root Bernstein et al., Anti-sense Peptides: A Critical Mini Review, J. Theoretical Biology, 190: 107-119 (1998). Markers derived by such antisense methods include Arg-Leu-Ala-His-Met-Tyr-Val-Gly-Lys-Thr (conjugated to KLH); Glu-Gly-Val-Tyr-Val-His-Pro-Val (conjugated to KLH); Glu-Thr-Met-Lys-Leu-Val-Thr-Gly-Ser-Pro-Ser (conjugated to KLH); and Glu-Glu-Thr-Gly-Val-Thr-Lys-Thr-Phe-Met-Thr-Asp-Lys.

In a preferred embodiment, the selected marker is compared to known databases of amino acid sequences. Accordingly, in a preferred embodiment, the methods for selecting a marker comprise the additional step of screening said candidate marker against a database of naturally occurring proteins and rejecting said candidate marker if said candidate marker is homologous to a protein in said database. Preferably, said candidate marker is rejected if it is more than about 50%, more preferably more than about 30%, more preferably more than about 20%, homologous to a protein in said database. Databases among these useful herein include the SwissProt and TrEMBL protein databases.

Nevertheless, it is important to realize in analyzing candidate markers that every candidate sequence will have some homology with some subset of known proteins. It was demonstrated by 1986 that every one of the 8,000 possible peptide triplets occurred in some protein in the protein database of that year. See, Doolittle, “redundancies in protein sequences,” In: G. Fasman, ed., Prediction of Protein Structure and the Principles of Protein Conformation. New York: Plenum, pp. 599-623 (1988). Many more protein sequences are known today. Thus, if for a given 16-mer peptide, which contains 14 sequential triplets, every one of those 14 triplets will occur somewhere in a protein database. No marker will ever be completely unique to the extent that it will have no homologous sequence with any naturally occurring protein. On the other hand, the knowledge that every possible peptide triplet does occur in the protein data base sets an important criterion for evaluating homology searches: markers are preferably sequences that approach the limit of having no more than a single sequential triplet homology with a single protein in the database. This criterion serves two purposes: first, it provides a testable goal for evaluating potential sequences; and second, peptides of length 9 to 16 that have little more than a sequential triplet homology with known proteins are very unlikely to be antigenically cross-reactive with naturally occurring proteins, since the substitution of even one or two amino acids in a sequence of 9 to 16 is often sufficient to eliminate antigenic cross-reactivity.

Preferably, markers are selected having adjacent amino acids having relatively low probability of occurring in nature. Many amino acids pairs and triplets (that is, one amino acid immediately followed by another in a sequence) are known to occur with either statistically significant overrepresentation or under representation in various protein data bases. A preferred method of identifying a marker comprises the additional step of screening amino acid pairs in the candidate marker against a database of naturally occurring amino acid pairs, and rejecting said candidate marker if does not comprise at least one, preferably more than one, rare amino acid pair. A more preferred method comprises the additional step of screening amino acid triplets in the candidate marker against a database of naturally occurring amino acid triplets, and rejecting said candidate marker if does not comprise at least one, preferably more than one, rare amino acid triplet. Such databases include those known in the art, such as disclosed in Cserzo et al., “Regularities in the primary structure of proteins,” Int J Peptide Protein Res 34: 184-195. (1989); and Gutman et al., “Nonrandom utilization of codon pairs in Escherichia coli,” Proc. Natl. Acad. Sci. USA 86: 3699-3703. (1989); and Doolittle, supra.

Although the methods of selecting markers described above are, in one embodiment, directed to peptide markers consisting of the common amino acids, similar methods may be used to make markers comprising other naturally and non-naturally occurring amino acids, non-natural linkages, and variants. Such methods include those described herein, wherein one or more amino acids are substituted with non-common or non-naturally occurring amino acids or variants, or wherein naturally occurring linkages are substituted with non-naturally occurring linkages. As will be appreciated by one of skill in the art, such methods for identifying peptoids or antisense peptides may be modified such that, for example, the attribute score and the average aggregate score of the candidate peptoid marker may be higher than the threshold levels discussed above with respect to peptide markers. Accordingly, such methods may be used for identifying markers consisting of the common amino acids, markers comprising other naturally occurring amino acids, markers comprising naturally occurring amino acids and non-naturally occurring amino acids, and markers consisting of non-naturally occurring amino acids.

Preferably a candidate marker is subjected to testing to determine whether it is antigenically dissimilar to other antigens which subjects to whom the marker is administered are likely to be exposed, including such antigens associated with infectious agents, foods, medicines, and autoimmune conditions. As referred to herein, “antigenically dissimilar” means that the antibodies produced by subjects to the marker may be distinguished from antibodies produced by subjects to such other antigens, using test methods to be used in the screening of subjects receiving the marker. Such testing may include in vitro and in vivo methods among those known in the art for detecting antibody cross-reactivity, including those discussed herein. Such methods include determining cross-reactivity of a candidate marker with antisera for infectious agents and antisera for hormones. Such methods including cross-reactivity of a candidate marker with other candidate markers. Preferably, a marker is selected from candidate markers having no or insignificant cross-reactivity in such tests.

Preferably, a candidate marker is tested in animal models of antigenicity. Markers for use with humans are also preferably subjected to appropriate clinical testing to ensure safety and to ensure that the marker is not antigenically similar to other antigens to which humans are likely to be exposed, including infectious agents, foods, medicines and antigens associated with autoimmune conditions. Also the antibodies elicited by the marker are preferably tested to ensure they are detectable using conventional antibody detection methods.

Accordingly, the present method provides methods of selecting a marker for administration to a human or animal subject, comprising
(a) identifying a candidate marker having from about 8 to about 100 amino acid residues;
(b) testing said marker to determine its antigenic dissimilarity with antigens to which the subject is likely to be exposed; and
(c) selecting said candidate marker if it antigenically dissimilar to said antigens.
   Preferably, such identifying comprises methods for identifying candidate markers described above. Preferably, such testing comprises a plurality of in vitro and in vivo tests, preferably including clinical testing among subjects of the species to whom the marker is to be administered. In such methods, the identification of antigens “to which the subject is likely to be exposed” is made using generally accepted principles of immunology, based on a reasonable determination of antigens found in the population of subjects to whom the marker is to be administered, and the desired specificity for marker screening (e.g., considering the need to avoid false positive test results while screening a population of subjects).

Candidate markers of the present invention include the following peptides, the sequences of which are included in the attached Sequence Listing.

| | | | |
|---|---|---|---|
| SEQ | | Average | Average |
| ID | | Attribute | Aggregate |
| NO: | Sequence | Score | Score |

| | | | |
|---|---|---|---|
| 1 | His-Trp-Arg-Trp-Arg-Met- | 6.1 | 3.7 |
| | Arg-Met-His-Trp-His-Met- | | |
| | Trp-Arg-Gln-Arg-Met-Trp | | |
| 2 | His-Trp-His-Trp-His-Trp- | 4.4 | 2.0 |
| | His-Trp-His-Trp-His-Trp- | | |
| | His-Trp-His-Trp-His-Trp | | |
| 3 | His-Trp-Arg-His-Trp-Met- | 5.5 | 3.3 |
| | His-Trp-Gln-His-Trp-Tyr- | | |
| | His-Trp-Cys-His-Trp-Phe | | |
| 4 | Tyr-His-Met-Trp-Tyr-His- | 5.3 | 2.5 |
| | Met-Trp-Tyr-His-Met-Trp- | | |
| | Tyr-His-Met-Trp | | |
| 5 | Trp-Cys-Met-His-Thr-Phe- | 8.0 | 6.5 |
| | Trp-Cys-Met-His-Thr-Phe | | |
| 6 | Trp-Pro-His-Asp-Met-Cys- | 7.5 | 6.2 |
| | Trp-Phe-His-Asp-Met-Cys | | |
| 7 | Arg-Arg-Tyr-Phe-Met-Tyr- | 6.9 | 4.6 |
| | Arg-Arg-Tyr-Phe-Met-Tyr- | | |
| | Arg-Arg-Tyr-Phe-Met-Tyr- | | |
| | Arg-Arg | | |
| 8 | Tyr-Phe-Met-Tyr-Arg-Arg- | 6.9 | 4.5 |
| | Tyr-Phe-Met-Tyr-Arg-Arg | | |
| 9 | His-Trp-Arg-Trp-Arg-Met- | 6.2 | 3.8 |
| | Arg-Met-His-Trp-Arg-Trp- | | |
| | Arg-Met-Arg-Met | | |
| 10 | Asp-Trp-Phe-His-Asp-Trp- | 7.3 | 6.0 |
| | Phe-His-Asp-Trp-Phe-His- | | |
| | Asp-Trp-Phe-His | | |
| 11 | Tyr-Phe-Met-Tyr-Arg-Arg- | 7.1 | 4.7 |
| | Tyr-Phe-Met-Arg-Arg | | |
| 12 | His-Trp-Arg-Trp-Arg-Met- | 6.2 | 3.8 |
| | Arg-Met | | |
| 13 | Trp-Pro-His-Asp-Met-Cys- | 6.4 | 5.3 |
| | Trp-Pro | | |
| 14 | His-Arg-Asn-Tyr-Phe-His- | 6.7 | 4.6 |
| | Arg-Asn-Tyr-Phe | | |
| 15 | Trp-Phe-Tyr-Met-Tyr-Met- | 7.1 | 4.6 |
| | Tyr-Phe-Trp-Phe-Tyr-Met- | | |
| | Tyr-Phe | | |
| 16 | Arg-Leu-Ala-His-Met-Tyr- | 10.1 | 10.1 |
| | Val-Gly-Lys-Thr | | |
| 17 | Glu-Gly-Val-Tyr-Val-His- | 10.8 | 12.1 |
| | Pro-Val | | |
| 18 | Glu-Thr-Met-Lys-Leu-Val- | 12.8 | 14.7 |
| | Thr-Gly-Ser-Pro-Ser | | |
| 19 | Glu-Glu-Thr-Gly-Val-Thr- | 12.6 | 13.5 |
| | Lys-Thr-Phe-Met-Thr-Asp- | | |
| | Lys | | |
| 20 | Trp-Phe-Tyr-Met-Tyr-Phe- | 7.2 | 4.8 |
| | Trp-Phe-Tyr-Met-Tyr-Phe | | |

In one embodiment, candidate markers are selected from the group consisting of SEQ ID NOs 1, 2, 3, 4, 5, 6, 7, 8, 10, 11, 12, 13, 15, 16, 17, 18, 19, 20, and mixtures thereof. In one embodiment, candidate markers are selected from the group consisting of SEQ ID Nos 8, 13, 14, 16, 20, and mixtures thereof, preferably consisting of 8, 13, 20, and mixtures thereof.
Synthesis of Markers:

The markers of the present invention may be made by methods including those well known in the art. In one embodiment, markers are synthesized in vitro using standard synthetic methods well known in the art. For example, peptides for use in this invention may be synthesized by known methods, such as those described in Fields, ed., Methods in Enzymology, Vol. 289 (Academic Press, New York, 1997). In non-limiting examples, two different solid state synthesis methods are widely used. They are designated Boc and Fmoc, according to the abbreviation for the amino protecting group used. Peptides may also be synthesized using the Merrifield solid-phase synthesis method, where amino acids are sequentially added to a growing amino acid chain. See, Merrifield, J. Am. Chem. Soc. 85:2149 (1963). Peptides may also be made using programmable peptide synthesizers which are commercially available and capable of performing synthesis steps automatically with good reliability and reproducibility. Equipment for automated synthesis of peptides is commercially available from suppliers such as Applied Biosystems, Foster City, Calif., U.S.A.

In another embodiment, markers are made biosynthetically, using recombinant techniques, in cultured host cells. In one such embodiment, a suitable host cell is transfected with an expression vector that comprises a polynucleotide sequence that encodes the marker, wherein the expression vector drives expression of the marker in the cell. Host cells among those useful herein include E. coli, yeast, insect cell lines, such as Spodoptera or Trichoplusia, and mammalian cell lines, such as CHO, COS, and NS- 1.

Expression vectors and means for making expression vectors useful herein include those well known in the art. A polynucleotide coding for a vaccination marker is preferably linked operatively to an enhancer-promoter. A promoter is a region of a DNA molecule typically from zero to ten nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer promoter is connected to a coding sequence in such a way that the transcription for that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alia upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

An enhancer-promoter used in a vector construct of the present invention can be any enhancer promoter that drives expression in a target cell. For example, the human cytomegalovirus (CMV) immediate early gene promoter can been used to result in high-level expression of a gene. However, the use of other viral or mammalian cellular promoters including those well known in the art is also suitable to achieve expression of the gene product provided that the levels of expression are sufficient to achieve a physiologic effect. Exemplary and preferred enhancer-promoters are the CMV promoter, the Rous sarcoma virus (RSV) promoter and the muscle-specific creatine kinase (MCK) enhancer. By employing an enhancer-promoter with well-known properties, the level and pattern of gene product expression can be optimized.

A polynucleotide of a vector construct is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in an adenovirus vector construct of the present invention preferably comprises a polyadenylation signal of SV40 or the protamine gene.

Polypeptoids and peptoids comprising amino acids other than the common amino acids may also be made biosynthetically, using recombinant techniques in cultured host cells. One such method comprises the creation of tRNA which codes for unused codons and utilizes an amino acid other than a common amino acid. Such orthogonal tRNA is then introduced into a host cell having genetic material, which is naturally present or added, that contains the codon for the tRNA. The peptide or peptoid comprising the non-common amino acid is then expressed and isolated for use as a marker. Such methods of producing orthogonal tRNA and peptides comprising non-natural amino acids are described in Wang L., et al., “Expanding the Genetic Code of Escherichia coli,” Science, 292, 498-500 (2001); and Mehl et al., “Generation of a Bacterium with a 21 Amino Acid Genetic Code,” J. Am. Chem. Soc., 125(4), 935-939 (2003).

The expressed peptides and peptoids are isolated in substantially pure form. Such purification is achieved using methods including those known in the art, such as ammonium sulfate fractionation, SDS-PAGE electrophoresis, and affinity chromatography.

Compositions:

The present invention provides antigenic marker compositions for administration to a human or other animal subject, comprising:

(a) an antigenic marker; and

(b) a pharmaceutically-acceptable carrier;

preferably wherein said marker does not elicit a vaccination immune response in said subject. In one embodiment, compositions of this invention comprise a single antigenic marker. In another embodiment, compositions of this invention comprise a plurality of (i.e., two or more) antigenic markers, wherein the immune response elicited by each of said antigenic markers is distinct from the immune response elicited the other markers in the composition. It will be appreciated that use of compositions comprising a plurality of antigenic markers will allow for identification of a greater number of characteristics in a population of subjects using relatively fewer unique individual markers. Preferably, such compositions comprise from 2 to 10, more preferably from 4 to 8, more preferably 5 or 6, antigenic markers. In addition use of compositions comprising multiple markers may also provide enhanced security and identification by increasing the number of marker combinations and, therefore, the amount of “decoding” necessary.

Also, optionally, the marker composition may comprises other antigenic materials. In a preferred embodiment, the composition comprises a vaccination antigen. As referred to here, a “vaccination antigen,” is a material which, when administered to a human or other animal subject, elicits an immunological response, different from that of the antigenic marker, that creates an immunity in the subject to an infectious diseases or other disorder. As referred to herein, an immunological response that is “different from that of the antigenic marker” is the production of antibodies, by a subject after administration of the vaccination antigen, that are distinguishable using conventional testing methods from the antibodies produced after administration of the antigenic marker. Infectious diseases for which vaccination antigens are used include those caused by infection with Salmonella, Shigella, Klebsiella, Enterobacter, Serratia, Proteus, Yersinia, Aeromonas, Pasteurella, Pseudomonas, Actineobacter, Moraxella, Flavobacterium, Bordetella, Actinobacillus, Neisseria, Brucella, Haemophilus, Bacillus anthracis, Escherichia coli, Chlostridium tetani, Corynebacterium diphtheriae, Vibrio cholerae, Clostridium perfringens, HIV, herpes, adenoviruses, rhinoviruses, hepatitis viruses, Epstein-Barr virus, cytomegalovirus, feline leukemia virus, canine distemper virus, aphthovirus, Mycobacterium leprae, Treponema pallidum, Chlamydia, Candida, and Pneumocystis.

Vaccination antigens among those useful herein are known in the art. Vaccination antigens useful herein include those derived from living organisms; intact or non-living organisms; subcellular fragments; toxoids; recombinant DNA-based antigens or anti-idiotypes (e.g., a cloned and expressed gene or naked DNA); synthetic antigens, and combinations thereof In one embodiment, the vaccination antigen is derived from natural or attenuated organisms, which may be viral or bacterial. In another embodiment, the antigen is a capsular polysaccharide, surface or internal antigen. The antigen may be modified by reaction, for example, with a cross-linking agent, such as a glutaraldehyde or another dialdehyde. In one embodiment, the vaccination antigen is a primary vaccine; in another embodiment, the vaccination antigen is a booster vaccine.

In one embodiment, the vaccination antigen comprises an organism that has been killed, e.g., by use of formalin. Such vaccination antigens include those referred to in the art as “inactivated” or “killed” vaccines, such as typhoid vaccine and the Salk poliomyelitis vaccine.

In another embodiment the vaccination antigen consists essentially of an antigenic part or parts of the disease causing organism, for example the capsule, the flagella, or part of the protein cell wall. Such vaccination antigens include those known in the art as “acellular vaccines,” such as the Haemophilus influenzae B (HiB) vaccine. Acellular vaccines exhibit some similarities to killed vaccines: neither killed nor acellular vaccines generally induce the strongest immune responses and may therefore require a “booster” every few years to insure their continued effectiveness. In addition, neither killed nor acellular vaccines can cause disease and are therefore considered to be safe for use in immunocompromised patients.

In another embodiment, the vaccination antigen comprises an “attenuated” or weakened live microorganism by aging it or altering its growth conditions. Examples of attenuated vaccines are those that protect against measles, mumps, and rubella. Immunity is often lifelong with attenuated vaccines and does not require booster shots.

In another embodiment, the vaccination antigen is made from toxins, such as by treating the toxin with aluminum or adsorbing it onto aluminum salts to decrease it's harmful effects. Such antigens are called a “toxoids.” Examples of toxoids are the diphtheria and the tetanus vaccines. In one embodiment, the toxoid is administered with an adjuvant to increase the immune response. For example, the diphtheria and tetanus vaccines are often combined with the pertussis vaccine and administered together as a DPT immunization. The pertussis acts as an adjuvant in this vaccine. When more than one vaccine is administered together it is called a “conjugated vaccine.” Toxoid vaccines often require a booster every ten years.

In another embodiment, the vaccination antigen comprises an organism which is similar to the virulent organism but that does not cause serious disease. An example of this type of vaccine is the BCG vaccine used to protect against Mycobacterium tuberculosis, which comprises an attenuated strain of Mycobacterium bovis and requires boosters every 3 to 4 years.

In another embodiment, genetic engineering techniques are used to produce “subunit vaccines” which use only the parts of an organism yet which stimulate a strong immune response. Such vaccination antigens are made by isolating the gene or genes which code for appropriate subunits from the genome of the infectious agent. This genetic material is then placed into bacteria or yeast host cells which produce large quantities of subunit molecules by transcribing and translating the inserted foreign DNA. The subunit molecules are then isolated, purified, and used as a vaccine. Hepatitis B vaccine is an example of this type of vaccine.

Vaccination antigens among those useful herein for human subjects include vaccines for adenovirus; anthrax; tuberculosis (e.g., BCG; Bacillus Calmette-Gruerin vaccine); Chagas' disease; cholera; E. coli; diphtheria toxoid; combinations of diphtheria and tetanus toxoids; combination of diphtheria and tetanus toxoids and acellular pertussus (DTaP); combinations of diphtheria and tetanus toxoids and whole cell pertussis; combinations of diphtheria and tetanus toxoids and pertussis and Haemophilus influenzae b (Hib) conjugate; Hib conjugate, with diphtheria, meningococcal and tetanus conjugates; hepatitis A; hepatitis B; hepatitis C; influenza virus; HIV; Japanese encephalitis virus; malaria; measles, combination of measles and mumps; combination of measles, mumps and rubella; meningococcal, Group A, meningococcal, Group B, meningococcal, Groups A and C, meningococcal, Groups A, C, Y and W-135, mumps virus, onchoceriasis; pertussis (acellular), pertussis (whole cell); plague; pneumococcus; Epstein-Barr virus; poliovirus, inactivated; poliovirus, live (attenuated); rabies; rhinovirus; rubella; schistosomiasis; smallpox (vaccinia); staphylococci; group A streptococci; tetanus; typhoid; trypanosomiasis; varicella (chickenpox); vole bacillus (for tuberculosis); and yellow fever vaccines. Vaccination antigens among those useful herein for non-human animal subjects include those for E. coli, feline leukemia, foot-and-mouth disease, and canine distemper. In one embodiment, the vaccination antigen creates an immune response in the subject to whom it is administered that is substantially indistinguishable from the immune response created by the organism against which the vaccination is effective. Preferred vaccination antigens include those for tuberculosis, hepatitis C, Epstein-Barr virus, cytomegalovirus, HIV, E. coli, feline leukemia, foot-and-mouth disease, and canine distemper.

In one embodiment, the immune response elicited by the vaccination antigen is substantially indistinguishable from the immune response elicited by the organism against which the vaccination if effective. As referred to herein, such a “substantially indistinguishable” response is the production of antibodies by a subject, after administration of the vaccination antigen, that cannot be distinguished, using conventional antibody detection methods, from the antibodies produced by infection with the organism against which the vaccination antigen is effective.

In a preferred method of this invention, such compositions comprising a marker and a vaccination antigen facilitate the identification of subjects having been vaccinated as opposed to having a naturally induced immune response due to the presence of the naturally occurring antigen. When the biomarker is used in conjunction with a vaccination antigen, the response created due to the vaccination antigen is substantially indistinguishable from the immune response created by the organism had the vaccination antigen been administered without the biomarker. Use of the biomarker in conjunction with the vaccination antigen produces an immune response that has relatively the same potency, effectiveness and longevity of the wild-type vaccine. The subject who has been administered a vaccine antigen in conjunction with a biomarker and the subject that has only been administered a wild-type vaccine are both afforded the same level of protection against the disease. For example, in one embodiment of the invention, the delivery of a Mycobacterium, the human immunodeficiency virus (HIV), the feline leukemia virus (FLV) and foot-and-mouth virus produces antigenic responses that raise the antibodies to Mycobacterium, HIV, FLV and foot-and-mouth virus, respectively. The subjects who get the vaccination delivered with a biomarker and the subject who is vaccinated without the use of the biomarker are afforded the same level of protection from disease.

Preferably, the compositions of this invention, comprising a marker and a vaccination antigen, are tested to ensure that the marker does not substantially reduce the immune response to the vaccination markers. Test methods for determining the effectiveness of the vaccination marker include these well known in the art.

In one embodiment, the compositions of this invention contain a single vaccination antigen. In another embodiment, the compositions of this invention comprise two or more vaccination antigens. In one such embodiment, the composition comprises two vaccination antigens that are typically delivered individually, such as the Hepatitis A and Hepatitis B vaccines. Alternatively, the first and second antigen can provide protection against distinct diseases for which a vaccine is traditionally delivered simultaneously such as the DTaP vaccine for diphtheria, tetanus and pertussis.

The composition optionally comprises an adjuvant to enhance the immune response to the antigenic marker. Adjuvants among those useful herein include aluminum hydroxide; aluminum in combination with 3-0 deacylated monophosphoryl lipid A; aluminum phosphate; N-acetyl-muramyl-L-threonyl-D-isoglutamine; N-acetyl-normuramyl-L-alanyl-D-isoglutamine; N-acetylmuramyl-L-alanyl-D-isoglutamyl-L-alanine 2 (1′dipalmitoyl-sn-glycero-3-hydroxyphosphoryloxy) ethylamine; monophosphoryl lipid A (MPA); trehalose-6,6-dimycolate (TDM), cell wall skeleton (CWS); RIBI, comprising a combination of MPA, TDM and CWS with detoxified endotoin, in a 2% squalene/TWEEN 80/emulsion (sold by ImmunoChem Research Inc., Hamilton, Mont., U.S.A.); Stimulon (sold by Cambridge Bioscience, Worchester, Mass., U.S.A.); MF 57 (sold by Chiron); SAF-1 (sold by Syntex); Complete Freund's Adjuvant (CFA); and Incomplete Freund's Adjuvant (IFA); and mixtures thereof. In some embodiments, the marker itself enhances immune response, and is useful as an adjuvant for the vaccination antigen. Thus, the present invention also provides methods for enhancing the immune response of a human or animal subject to a vaccination antigen, comprising co-administering to said subject said vaccination antigen and a marker.

Pharmaceutically-acceptable carriers useful in the compositions of this invention include those well known in the art. Specific carrier components will depend upon such factors as the route of administration and the physical and chemical characteristics of the marker and any other materials to be administered with the marker. Compositions among those useful herein include those that are known in the art for the administration of vaccination antigens.

A preferred route of administration is injectable, preferably by parenteral injection subcutaneously or intramuscularly. Injectable compositions useful herein include liquid solutions or suspensions. Suspensions include those where the marker is emulsified or encapsulated in liposomes. Compositions may optionally comprise diluents and excipients that are pharmaceutically acceptable and compatible with the formulation. Such excipients include water, saline, dextrose, glycerol, ethanol, propylene glycol, ethyl oleate, pyrrolidone, sesame oil, wetting or emulsifying agents, pH buffering agents, and mixtures thereof. In one embodiment, the composition is provided for use in liquid form, and is placed into a sterile container after formulation which is then sealed and stored at low temperature (e.g., at about 40° C.). In another embodiment, the composition is provided in solid form, such as through lyophilization of a liquid composition. The solid form is then reconstituted by mixing with a suitable liquid carrier (e.g., saline) prior to injection.

The concentration of antigenic marker in the injectable composition can be varied over a broad range. Preferably, the concentration of antigenic marker in the composition is from about 0.2 μg/ml to about 200 μg/ml, more preferably from about 5 μg/ml to about 50 μg/ml. A preferred composition comprises antigenic marker in a concentration of about 15 μg/ml/ml.

Other routes of administration useful in the methods of this invention include oral, nasal, rectal, and topical. Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents. Preferred carriers for oral administration include gelatin, propylene glycol, cottonseed oil and sesame oil.

The compositions of this invention can also be administered topically to a subject, i.e., by the direct laying on or spreading of the composition on the epidermal or epithelial tissue of the subject. Such compositions include, for example, lotions, creams, solutions, gels and solids, and may, for example, be locally or systemically administered transdermally or by intranasal, pulmonary (e.g., by intrabronchial inhalation), ocular, or other mucosal delivery. Suitable carriers for topical administration on skin preferably remain in place on the skin as a continuous film, and resist being removed by perspiration or immersion in water. Generally, the carrier is organic in nature and capable of having dispersed or dissolved therein the antigenic marker compounds. The carrier may include pharmaceutically-acceptable emollients, emulsifiers, thickening agents, and solvents.

Formulations suitable for mucosal administration by inhalation include compositions of the adrenergic and complement compounds in a form that can be dispensed by inhalation devices among those known in the art. Such formulations preferably comprise liquid or powdered compositions suitable for nebulization and intrabronchial use, or aerosol compositions administered via an aerosol unit dispensing metered doses. Suitable liquid compositions comprise the active ingredient in an aqueous, pharmaceutically acceptable inhalant solvent, e.g., isotonic saline or bacteriostatic water. The solutions are administered by means of a pump or squeeze-actuated nebulized spray dispenser, or by any other conventional means for causing or enabling the requisite dosage amount of the liquid composition to be inhaled into the lungs.

Suitable powder compositions include, by way of illustration, powdered preparations of the active ingredients thoroughly intermixed with lactose or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. Aerosol formulations preferably include propellants, surfactants and co-solvents and may be filled into conventional aerosol containers that are closed by a suitable metering valve.

Methods of Use:

The present invention provides methods of marking a human or other animal subject comprising the administration to the subject of a safe and effective amount of an antigenic marker. In particular, such methods for marking an individual subject having a given characteristic in a population of animal subjects, comprise:

(a) identifying an individual subject having said characteristic; and

(b) administering to said individual a marker composition comprising an antigenic marker.

The present invention also provides methods for tracking individual animal subjects having a characteristic in a population of subjects having a plurality of characteristics, comprising:

(a) providing a marker composition which is associated with said characteristic, wherein said composition comprises an antigenic marker that does not raise a vaccination immune response in said subjects;

(b) administering said marker composition to subjects having said characteristic; and

(c) screening subjects in said population to identify subjects to whom said marker composition has been administered.

In a preferred embodiment, such methods comprise administering a plurality of antigenic markers to the individual subjects having the given characteristic. Preferably, from 2 to 10, more preferably from 4 to 8, more preferably 5 or 6 markers, are administered.

Such methods of this invention comprise eliciting unique antibodies in an animal subject, comprising administering to said subject a composition comprising an antigenic marker, wherein said marker does not raise a vaccination immune response in said subject. In one embodiment, the subject is a human subject. In another embodiment, the subject is non-human animal, preferably an animal having economic, scientific or emotional significance. Non-human animals with which the methods of this invention may be used include agricultural livestock, such as cattle, pigs, chickens and other fowl, sheep, and goats; domestic companion animals, such as household pets (e.g., dogs and cats), show animals, racing animals (e.g., horses); laboratory animals (e.g., mice, rats, guinea pigs); and other animals such as may be found in the wild (e.g., endangered species) or in zoos.

The antigenic markers of this invention are administered in a manner compatible with the antigenic marker, vaccination antigen (if any), and the dosage formulation. As referred to herein, a “safe and effective amount” of an antigenic compound is an amount that is sufficient to elicit an antigenic response in human or other animal subject to whom the compound is intended to be administered, without undue adverse side effects (such as toxicity, irritation, or allergic response), commensurate with a reasonable benefit/risk ratio when used in the manner of this invention. The specific safe and effective amount of the antigenic compound will, obviously, vary with such factors as the particular intended utility of the method (e.g., for vaccination of the subject), the taxonomic group of the subject (e.g., human, primate, etc.), the physical condition of the subject, the capacity of the individual's immune system to mount an effective immune response, the nature of concurrent therapies (if any), the specific antigenic compound used, the specific route of administration and dosage form, the carrier employed, and the desired dosage regimen. Preferably, the marker is administered in a dosage range of from about 0.01 μg to about 1000 μg per dose, preferably from about 0.1 μg to about 250 μg per dose; more preferably from about 20 μg/ml to about 100 μg/ml per dose. The marker may be administered in a single dose, or in a multiple dose schedule. A multiple dose schedule is one in which a primary course of vaccination comprises from 1 to 10 separate doses, followed by other doses given at subsequent time intervals required to maintain and or reinforce the immune response, for example, at 1 to 4 months for a second dose, and if needed, subsequent dose(s) after several months. The antigenic marker may be administered in conjunction with other immunoregulatory agents, for example, immunoglobulins.

The present invention also provides methods for identifying a subject having a given characteristic in a population of animal subjects. Such methods comprise:

(a) administering to individual subjects having said characteristic an antigenic marker that, preferably, does not raise a vaccination immune response in said subject; and

(b) screening said population to identify individuals having said characteristic by the presence of an antibody to said marker in said individual.

In such methods, the screening step is typically conducted at a time significantly after the administering step, which may be days, months or years later. In such methods, the population typically comprises individuals who have the characteristic, and individuals who do not have the characteristic. In one embodiment, the subject has one characteristic of interest. In another embodiment, the individual subject has two or more characteristics. In a preferred embodiment, such methods comprise administering a plurality of antigenic markers to the individual subjects having the given characteristic. Preferably, from 2 to 10, more preferably from 4 to 8, more preferably 5 or 6, markers are administered. In one embodiment, the such methods comprise administering a single composition comprising a plurality (i.e., two or more) markers. In another embodiment, such methods comprise administering two or more compositions, each of which comprise a plurality of markers, thereby increasing the total number of distinct markers administered. Such multiple compositions may be administered concurrently, or may be administered individually over a period of time.

As referred to herein, a “characteristic” is any trait, quality, property or other parameter which may be used to differentiate one subject from at least one other subject in a population of subjects. The characteristic preferably has significance to a user or group of users, such as physical, social, emotional, or commercial significance. Such characteristics include vaccination status, ownership (e.g., for branding of cattle), point of origin (e.g., for tracking the handling of livestock through commercial processing), location (e.g., for tracking the movement of wild animals in nature), and lineage (e.g., for identification of purebred animals). Tracking is performed by administering an antigenic marker to the individual to produce antibodies that are unique to the antigenic marker and then screening the population to identify the individual based on the presence of the marker antibodies. The antibody to the marker elicited in the subject, similar to a vaccination-induced antibody, is preferably detectable throughout the life of the animal subject. For example, in the case of livestock, the presence of a biomarker can be detected through meat processing until the flesh of the animal is cooked (when the antibody structure is denatured).

In a preferred embodiment, the characteristic is the state of vaccination of the subject for one or more diseases. Accordingly, the present invention provides methods of immunizing an individual at risk of being infected by a pathogen, comprising administering to said individual a vaccine comprising:

(a) a vaccination antigen; and

(b) an antigenic marker;

wherein said antigenic biomarker raises an antibody in said individual that is different from antibodies raised by said vaccination antigen. The present invention also provides methods for identifying vaccinated subjects, to whom a vaccination antigen has been administered, in a population of animal subjects, comprising:

(a) administering to said vaccinated subjects an antigenic marker that does not raise a vaccination immune response in said subject; and

(b) screening said population to identify said vaccinated individuals by the presence of an antibody to said marker in said individual.

Preferably, the antigenic marker is administered to the individual concurrently with the vaccination antigen, preferably in a composition of this invention. In such methods, the screening step is typically conducted at a time significantly after the administering step, which may be months or years later. In such methods, the population typically comprises individuals who are vaccinated subjects and individuals who are not vaccinated subjects. In some embodiments, the population additionally comprises individuals who have been infected with the pathogen for which the vaccination is effective.

The screening step comprises any method for determining the presence of antibodies to the vaccination antigen in individual subjects in a population of subjects in which some subjects have been administered the vaccination antigen. The presence of antibodies may be detected in vivo, by testing the subjects directly, or in vitro by testing tissues taken from the subjects. In a preferred embodiment, the method comprises testing tissues taken from the subject. Such tissues useful herein include blood, saliva, urine, milk, skin, or hair or other tissues in which antibodies may be found. In one embodiment, the tissue is blood. In another embodiment, for tracking agricultural livestock, the tissue may be meat from the livestock subject obtained during processing.

The screening step preferably comprises testing the tissue for the presence of antibodies for the antigenic marker associated with the characteristic. As referred to herein, the marker “associated with the characteristic” is the marker that, during the administering step of methods of this invention, was administered to subjects having the characteristic. Antibody tests among those useful in the methods of this invention include those well known in the art. Such antibody detection methods include immunoblotting, immunoprecipitation, immunohistochemistry, radioimmunoassay, Western Blot, chemiluminescent assays, bioluminescent assays (e.g., luciferase-luciferin), precipitation assays (e.g. avidin-biotin), and enzyme immunoassays such as ELISA (enzyme-linked immunosorbent assay) and ELIFA (enzyme-linked immunoflow assay). ELISA and ELIFA are preferred assays useful herein. Depending on the type of biological sample collected from the subject and the test method used, the biological sample may require purification before the test is administered.

The present invention also provides identification kits, for identifying animal subjects having been administered a target antigenic marker, comprising:

(a) the antigenic marker or antigenic equivalent thereof; and

(b) an indicator which provides a signal when a tissue sample from said subject is contacted with said marker.

As referred to herein, the “target” antigenic marker is a marker which has been administered to subjects having a particular characteristic (e.g., having been administered a particular vaccination antigen) in a population of subjects to be screened using the kit. In some embodiments, the kit is for use in screening tissue samples for the presence of a single target marker. In another embodiment, the kit is for use in screening tissue samples for the presence of antibodies for two or more target markers.

The marker in the kit is antigenically indistinguishable from the target marker, such that the antibodies elicited by the target marker in a subjects to whom it is administered bind with the antigenic marker in the kit. In one embodiment, the antigenic marker in the kit is identical to the target marker. In another embodiment, the antigenic marker in the kit is an antigenic equivalent of target marker. As referred to herein, an “antigenic equivalent” is a peptide or peptoid which binds with antibodies produced by the subject to whom the antigenic marker is administered, and which is a variant of the antigenic marker. A “variant,” as referred to herein, is a peptide or peptoid that differs from the antigenic marker only in conservative substitutions and/or modifications, such that the immunogenic properties of the marker are retained. Peptide variants preferably exhibit at least about 70%, more preferably at least about 90% and most preferably at least about 95% homology to the antigenic marker. In one embodiment, variants are identified by evaluating a modified peptide for the ability to generate antibodies that bind to the antigenic marker. Such modified sequences may be prepared and tested using, for example, the methods described herein with respect to antigenic markers. As used herein, a “conservative substitution” is one in which an amino acid is substituted for another amino acid that has similar properties, such that one skilled in the art of peptide chemistry would expect the secondary structure and hydropathic nature of the peptide to be substantially unchanged. In general, the following groups of amino acids represent conservative changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; and (5) Phe, Tyr, Trp, His. In one embodiment, variants also contain other modifications, including the deletion or addition of amino acids that have minimal influence on the antigenic properties, secondary structure and hydropathic nature of the peptide. For example, a peptide may be conjugated to a signal (or leader) sequence at the N-terminal end of the protein which co-translationally or post-translationally directs transfer of the protein. The peptide may also be conjugated to a linker or other sequence for ease of synthesis, purification or identification of the peptide (e.g., poly-His), or to enhance binding of the peptide to a solid support. For example, a peptide may be conjugated to an immunoglobulin Fc region.

The indicator may be any device or material which emits a perceptible signal to the user of the kit when an antibody for the target marker is contacted with the antigenic marker. In a preferred embodiment, the specific antibody that corresponds with the antigenic marker will bind to such marker and provide a signal to indicate the binding. In general, the antigenic marker or antigenic equivalent thereof is solubilized with an appropriate buffer and provided on a substrate, such as a plate or well. The tissue sample received from the subject is purified to obtain antibodies. Upon exposing the purified antibody serum to the antigen bound solid phase, the specific antibodies bind to the antigenic markers on the solid phase. Subsequently the solid phase is evaluated to determine the level of the signal provided by the indicator, either by emitting light, color, or an alternate type of signal. Indicators include luminescent particles (either chemical or biologically based), dye-like particles, a chemical reactive particles, and other types of indicators including known in the art. In another embodiment, alternate type binding mechanisms are used which provide a conjugate specific or unspecific type antibodies that act either as an intermediate to the antigen antibody provided on the solid phase.

Specific assay useful herein include those known to those of ordinary skill in the art for using peptides to detect antibodies in a sample. Methods adaptable for use herein are described in the following references: Harlow, et al., Antibodies: A Laboratory Manual, Cold Spring Harbor Laboratory (1988); Roitt et al., Immunology, 3^{rd} ed., Mosby (1993); PCT Publication WO 96/13590, Maertens et al., published May 9, 1996; PCT Publication WO 96/29605, Reed et al., published Sep. 26, 1996; U.S. Pat. No. 6,458,366, Reed et al., issued Oct. 1, 2002; and U.S. Pat. No. 6,458,922, Zrein, issued Oct. 1, 2002. In one embodiment, the antigenic marker or antigenic equivalent thereof is immobilized on a solid support to bind to and remove the antibody from the sample. The bound antibody is then detected using a detection reagent that contains a reporter group. Suitable detection reagents include antibodies that bind to the antibody/marker complex and free peptide labeled with a reporter group (e.g., in a semi-competitive assay). Alternatively, a competitive assay may be utilized, in which an antibody that binds to the peptide is labeled with a reporter group and allowed to bind to the immobilized antigen after incubation of the antigen with the sample. The extent to which components of the sample inhibit the binding of the labeled antibody to the peptide is indicative of the reactivity of the sample with the immobilized peptide.

The solid support may be any solid material known to those of ordinary skill in the art to which the antigen may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681, Jorgenson et al., issued Oct. 25, 1994. The antigenic marker or antigenic equivalent thereof is bound to the solid support using a variety of techniques including known to those of ordinary skill in the art. The term “bound” refers to both noncovalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the marker, in a suitable buffer, with the solid support for a suitable amount of time. The contact time varies with temperature, but is typically between about 1 to about 24 hours. In general, contacting a well of a plastic microtiter plate (such as polystyrene or polyvinylchloride) with an amount of peptide ranging from about 10 ng to about 1 μg, and preferably about 100 ng, is sufficient to bind an adequate amount of the marker. Covalent attachment of the marker to a solid support may generally be achieved by first reacting the support with a bifunctional reagent that will react with both the support and a functional group, such as a hydroxyl or amino group, on the marker. For example, the marker may be bound to supports having an appropriate polymer coating using benzoquinone or by condensation of an aldehyde group on the support with an amine and an active hydrogen on the peptide.

In a preferred embodiment, the assay is an enzyme linked immunosorbent assay (ELISA). This assay may be performed by first contacting the marker that has been immobilized on a solid support, commonly the well of a microtiter plate, with the sample, such that antibodies to the marker within the sample are allowed to bind to the immobilized marker. Unbound sample is then removed from the immobilized peptide and a detection reagent capable of binding to the immobilized antibody-peptide complex is added. The amount of detection reagent that remains bound to the solid support is then determined using a method appropriate for the specific detection reagent. More specifically, once the peptide is immobilized on the support as described above, the remaining protein binding sites on the support are typically blocked. Any suitable blocking agent known to those of ordinary skill in the art, such as bovine serum albumin or Tween 20™. (sold by Sigma Chemical Co., St. Louis, Mo., U.S.A.) may be used.

The immobilized peptide is then incubated with the sample, and antibody is allowed to bind to the antigen. The sample may be diluted with a suitable diluent, such as phosphate-buffered saline (PBS) prior to incubation. In general, an appropriate contact time (i.e., incubation time) is sufficient to detect the presence of antibody within a tissue sample, preferably sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient. Unbound sample may then be removed by washing the solid support with an appropriate buffer, such as PBS containing 0.1% Tween 20.

Detection reagent is then added to the solid support. An appropriate detection reagent is any compound that binds to the immobilized antibody-peptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups, biotin and colloidal particles, such as colloidal gold and selenium. The conjugation of binding agent to reporter group may be achieved using standard methods known to those of ordinary skill in the art. Common binding agents may also be purchased conjugated to a variety of reporter groups from commercial sources, such as Zymed Laboratories, San Francisco, Calif., U.S.A., and Pierce, Rockford, Ill., U.S.A.

The detection reagent is then incubated with the immobilized antibody-marker complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of antibodies in the sample, the signal detected from the reporter group that remains bound to the solid support is preferably compared to a signal that corresponds to a predetermined cut-off value. In one embodiment, the cut-off value is the average mean signal obtained when the immobilized marker is incubated with samples from an uninfected patient. In general, a sample generating a signal that is one ore more, preferably two or three, standard deviations above the predetermined cut-off value is considered positive. In an alternate preferred embodiment, the cut-off value is determined using a Receiver Operator Curve, according to the method of Sackett et al., Clinical Epidemiology: A Basic Science for Clinical Medicine, Little Brown and Co., 106 (1985). In this embodiment, the cut-off value is determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive.

In another embodiment, the assay is performed in a rapid flow-through or strip test format, wherein the marker is immobilized on a membrane, such as nitrocellulose. In the flow-through test, antibodies within the sample bind to the immobilized marker as the sample passes through the membrane. A detection reagent (e.g., protein A-colloidal gold) then binds to the antibody-marker complex as the solution containing the detection reagent flows through the membrane. The detection of bound reagent may then be performed as described above. In the strip test format, one end of the membrane to which marker is bound is immersed in a solution containing the sample. The sample migrates along the membrane through a region containing detection reagent and to the area of immobilized peptide. Concentration of detection reagent at the peptide indicates the presence of marker antibodies in the sample. Typically, the concentration of detection reagent at that site generates a pattern, such as a line, that can be read visually. The absence of such a pattern indicates a negative result. In general, the amount of marker immobilized on the membrane is selected to generate a visually discernible pattern when the biological sample contains a level of antibodies that would be sufficient to generate a positive signal in an ELISA, as discussed above. Preferably, the amount of marker immobilized on the membrane ranges from about 25 ng to about 1 μg, and more preferably from about 50 ng to about 500 ng. Such tests can typically be performed with a very small amount (e.g., one drop) of serum or blood from the animal subject.

Identification and Tracking Systems:

The present invention provides systems for identifying and tracking subjects having a characteristic in a population of subjects. Such systems comprise antigenic markers and methods of this invention. In particular, such systems comprise:

(a) a marker composition comprising an antigenic marker that, preferably, does not raise a vaccination immune response in the subject;

(b) a marking system, for administering said marker composition to the subjects having said characteristics; and

(c) a testing system, for screening subjects in said population to identify the subjects who have been administered said antigenic marker.

As referred to herein, a “marking system” is any system comprising equipment and processes by which subjects having a given characteristic are identified and administered a marker composition. Such a system uses compositions and methods of this invention. As referred to herein, a “testing system” is any system comprising equipment and processes by which a population of subjects may be tested to identify those individuals that have been administered an antigenic marker. In a preferred embodiment, tissue samples of the subjects are tested for the presence of antibodies to the antigenic marker that has been administered to subjects by use of the marking system.

Equipment and processes useful in the systems of this invention include computer hardware and software to facilitate the identification of individual subjects who have been administered marker compositions, devices for administering the compositions of this invention (e.g., syringes, if the composition is injectable), and analytical devices (such as a test kit of the present invention) to test tissue samples of subjects. In a preferred embodiment, the present invention provides a database for storing information regarding subjects to whom said marker composition has been administered. In one embodiment, the stored information comprises the identity of the said subjects and identification of the characteristic being tracked. In a preferred embodiment, the database is stored on one or more computer-readable media, for use with appropriate computer hardware and software. In one embodiment, the database resides on a central computer, for access by system users in a plurality of remote locations (e.g., by direct connection, modem, or internet connection). In a preferred embodiment, the present invention provides a database comprising:

(a) the composition of a marker composition comprising an antigenic marker, wherein, preferably, said antigenic marker does not elicit a vaccination immune response in said subjects; and

(b) identification of a characteristic with which said marker is associated.

In one embodiment, the database additionally comprises identification of one or more subjects to whom said marker has been administered.

System users include any individuals or other entities who track or identify subjects having a characteristic using marker compositions of this invention. In one embodiment, for tracking the vaccination status of subjects, such users include health care providers, insurance providers, vaccination manufacturers, and agencies or other organizations regulating vaccine registration and commercialization. In another embodiment, for tracking the ownership of animals, such users include animal owner associations, agricultural livestock processors, and agencies or other organizations regulating food supplies. In one embodiment, systems of this invention are used by a single group of users, wherein the characteristics being tracked are related (i.e., have a common attribute of significance to the user, such as tracking the administration of multiple vaccines by vaccination manufacturers or other health care providers). In another embodiment, systems of this invention are used by a plurality of groups of users, wherein the characteristics being tracked are unrelated (do not have a common attribute of significance).

Use of the compositions and methods of this invention preferably employ systems for regulating the selection of markers and their association with characteristics that are to be tracked. Accordingly, the present invention provides administration systems for tracking subjects in a population of subjects having a first characteristic and a second characteristic, wherein:

(a) said system associates a first antigenic marker with said first characteristic;

(b) said system associates a second antigenic marker with said second characteristic, wherein said second antigenic marker is antigenically unique relative to said first antigenic marker; and (preferably)

(c) said antigenic markers do not elicit a vaccination response in said subjects.

As referred to herein, “associates” refers to the identification, or assignment within the system, of a marker composition to a single characteristic or group of characteristics, such that the marker composition is not identified with, or assigned to, any other characteristic or group of characteristics. In one embodiment, the marker composition comprises a single antigenic marker. In another embodiment, the marker composition comprises two or more antigenic markers. In one embodiment, each characteristic is associated with a single marker composition. In one embodiment, the first and second characteristics are related. In another embodiment, the first and second characteristics are unrelated. Preferably, the population of subjects comprises multiple characteristics, each of which is associated with its own marker composition. In a preferred embodiment, such methods comprise administering a plurality of antigenic markers to the individual subjects having the given characteristic. Preferably, from 2 to 10, more preferably from 4 to 8, more preferably 5 or 6, markers are administered.

The following are non-limiting Examples of the compounds, compositions, systems, and methods of this invention.

### EXAMPLE 1

An antigenic marker is made comprising the peptide of SEQ ID NO:1, using an amino acid sequencer. The marker is then tested for antigenicity by emulsifying 15 mg of the peptide in 0.5 ml Freund's incomplete adjuvant and 0.5 ml sterile saline. About 0.3 ml of the emulsion is then inoculated subcutaneously into the back of a rat. The inoculation is repeated in the rat two and four weeks after the initial inoculation (in a regimen consistent with the administration of some vaccines). At week six, a 0.1 ml blood sample is drawn by tail vein bleed, diluted in 0.5 ml EDTA and spun down. The resulting serum is used for ELISA.

In the ELISA procedure, 0.1 mg of the marker is dissolved in 1.0 ml of sterile water and 0.05 ml introduced in the wells of an ELISA plate. The marker solution is allowed to incubate at room temperature for one hour, after which it is washed out three times with phosphate buffered saline with 0.1% Tween 20 (PBSTween). Polyvinyl alcohol (0.050 ml, 1% in sterile water) is added to each well as a blocking agent and allowed to incubate at room temperature for one hour. The PVA is washed out as above with PBSTween. The rat serum sample (0.050 ml) is added to the wells and allowed to incubate at room temperature for about one hour and then washed out as above. 0.050 ml goat-anti-rat HRP antibody diluted by 10,000 fold is then added to the wells and incubated for one hour before being washed out as above. 0.050 ml ABTS is then added to the wells and the plate is read with a visible light spectrophotometer at 5, 10, 20 and 40 minutes. A second set of wells is created without any marker, containing only PVA, serum, anti-rat antibody, and ABTS. This set of wells provides a control giving background readings. The absorbance of the control wells are subtracted from the absorbance of the antibody-containing wells on the plate. Antibody to the marker is considered to be present in the serum if the absorbance for a the test well is one or more standard deviations above the background reading.

### EXAMPLE 2

An injectable composition is made comprising the marker of SEQ ID NO:2, and a hepatitis C vaccine antigen. The composition is administered to health care providers, according to a standard protocol for administering heptatitis C vaccine. Several years later, one of the health care workers is tested for hepatitis C, revealing antibodies to the disease in his blood. The subject is then tested for the antibody to the marker Seq Id No 2, and antibodies are also found, proving that the subject was vaccinated for hepatitis C and has not been infected.

### EXAMPLE 3

A national antigenic branding program is established for cattle, administered by a cattle branding association. An individual cattle farmer contacts the association, and is provided 1000 doses of a marker composition of this invention, comprising marker SEQ ID NO:5 and SEQ ID NO:6. The association maintains a database, associating the composition and the specific combination of those two antigenic markers, with the identify of the farmer. The farmer inoculates his cattle with the marker composition. Several months later, two of his cattle are stolen. After investigation, the cattle are found at a livestock yard, having been presented for processing. Antibody testing of the cattle confirm that the cattle belong to the cattle farmer.

### EXAMPLE 4

A cattle farmer sells 500 cattle that have been inoculated with the marker composition of Example 3. Meat from some of the cattle are processed into ground beef, under conditions that result in contamination of the beef with E. coli. The beef is subsequently sold to distributors. The contamination is later discovered, and the contaminated beef is tested for marker compositions. Antibodies are detected, and the cattle branding association contacted to determine the origin of the beef. The association searches its database, and identifies the cattle farmer, who then identifies the processor for the beef.

### EXAMPLE 5

A national association for purebred dogs establishes a marker registry program. As part of the program, dog owners of a particular breed who want to claim official status must inoculate their dogs with marker compositions provided by the association. A German Shepherd owner wishes to certify his dog, and applies to the association. After confirming the dog's qualifications, the association provides the owner with a unique marker composition, and makes an entry in the association's database associating the marker composition with the owner, the dog, and its breed. The owner inoculates the dog with the marker composition.

Subsequently, the dog is entered into a dog show. The show organizers draw a blood sample from the dog, and analyze it for marker antibodies. The organizers access a database maintained by the association, and confirm the dog's status as being a purebred German Shepherd, and confirm the owner's identity.

Several years later, the dog is lost. The dog is found, without its tags, at an animal shelter. The shelter, as part of a routine screening process for found dogs, takes a blood sample and analyzes it for marker antibodies. The results are compared to the association's database, and the owner of the dog is identified.

### EXAMPLE 6

An association of licensed cheetah breeders establishes a program to identify animals that are legitimately bred, in order to detect and prevent illegal importation of cheetahs. As part of the program, each breeder is provided with a unique marker composition comprising five antigenic markers of this invention, and a database is created associating the marker composition with the identity of the breeder.

A law enforcement agency encounters an individual with a captive cheetah. A blood sample is obtained from the cheetah. The sample is screened for antibodies to markers in the association's database, and no markers are found. Based on this finding, the law enforcement agency further investigates the individual, and determines that he is importing wild cheetahs. The individual is then arrested and charged for violation of applicable laws.

### EXAMPLE 7

About ten milligrams of each of seven candidate marker peptides (set forth in Table 1) was conjugated to keyhole limpet hemocyanin (KLH) to boost antigenicity. An equal amount of each candidate marker was left un-conjugated in order to perform ELISA assays.

TABLE 1

Tested Candidate Markers

| | | |
|---|---|---|
| Candidate | SEQ ID | |
| No. | NO | Sequence |

| | | |
|---|---|---|
| RBM1 | 8 | Tyr-Phe-Met-Tyr-Arg-Arg-Tyr-Phe- |
| | | Met-Tyr-Arg-Arg |
| RBM2 | 14 | His-Arg-Asn-Tyr-Phe-His-Arg-Asn- |
| | | Tyr-Phe |
| RBM3 | 20 | Trp-Phe-Tyr-Met-Tyr-Phe-Trp-Phe- |
| | | Tyr-Met-Tyr-Phe |
| RBM4 | 16 | Arg-Leu-Ala-His-Met-Tyr-Val-Gly- |
| | | Lys-Thr |
| RBM5 | 17 | Glu-Gly-Val-Tyr-Val-Pro-Val |
| RBM6 | 9 | His-Trp-Arg-Trp-Arg-Met-Arg-Met- |
| | | His-Trp-Arg-Trp-Arg-Met-Arg-Met |
| RBM7 | 13 | Trp-Pro-His-Asp-Met-Cys-Trp-Pro |

The candidate markers were screened for cross-reactivity to polyclonal sera against a range of infectious agents and hormones. The sera used were HIV-1, HIV-gp120, HIV-gp41, CMV, HBV, HSV-1, HSV-2, M. tuberculosis, T. pallidum, angiotensin II, insulin, and glucagon. Three milligrams of each biomarker peptide was dissolved in 3.0 ml phosphate buffer (pH 7.0). Aliquots (0.1 ml) of these solutions were then added to the wells of Costar ELISA plates (96 well, round-bottom) and incubated at room temperature for three hours. The peptide solutions were washed out three times using a manual plate washer employing 0.4 ml pH 7.0 phosphate buffer with 0.1% Tween 20. Polyvinyl alcohol (PVA) solution (0.2 ml, saturated solution in phosphate buffer) was added to each ELISA plate well as a blocking agent and incubated for one hour at room temperature. Wells without markers were also coated with PVA, as controls. The PVA was washed out as above. The sera listed above were diluted 1/1000 in phosphate buffer. 0.1 ml of these antisera solutions were added to the ELISA plate wells and incubated for three hours at room temperature. If HRP-conjugated antisera were used, then the color reaction agent ABTS (Chemicon) was added (0.1 ml/well) and the color reaction read using a Spectromax scanning spectrophotometer at 410 nm, 30 minutes after addition of the ABTS. If the antisera were not HRP-conjugated, then an HRP-conjugated secondary antibody appropriate to the species of the antiserum was prepared at 1/1000 dilution in phosphate buffer; 0.1 ml added to each well; incubated for one hour; washed out as above; and then 0.1 ml of ABTS added and the color reaction read as above.

In order to test the antigenicity and possible cross-reactivity of various candidate markers and combinations (as set forth in Table 3), groups of three Lewis rats were each inoculated subcutaneously at the base of the tail with 1.0 mg of the each biomarker antigen-KLH conjugate in 0.1 ml sterile saline solution emulsified with 0.1 ml Freund's incomplete adjuvant. At fourteen to twenty-one days following inoculation, 0.1 ml blood was removed from each animal by tail vein bleed or, of the animals had been sacrificed, 1.0 ml was removed by heart puncture following euthanasia. Blood was collected in EDTA tubes. The red blood cells were spun down using an Ependorff centrifuge. The clear sera were collected and diluted to 1/100 in phosphate buffer and these solutions were used to carry out ELISA experiments as described above.

The cross-reactivity of the candidate markers to antisera against various infectious agents and hormones is set forth in Table 2.

TABLE 2
Cross-Reactivity Studies of Marker Candidates Versus Antisera

| | Candidate Biomarker | | | | | | |
|---|---|---|---|---|---|---|---|
| Sera | RBM1 | RMB2 | RBM3 | RBM4 | RBM5 | RBM6 | RBM7 |
| HIV-1 | − | − | − | − | − | − | − |
| HIV-gp120 | − | − | − | − | − | − | − |
| HIV-gp41 | − | − | − | − | − | − | − |
| CMV | − | − | − | − | − | − | − |
| HBV | − | − | − | − | − | − | − |
| HSV-1 | − | − | − | − | − | − | − |
| HSV-2 | − | − | − | − | − | − | − |
| M. tuberculosis | − | − | − | − | − | − | − |
| T. pallidum | − | − | − | − | − | − | − |
| angiotensin II | − | − | − | − | + | − | − |
| insulin | − | − | − | − | − | − | − |
| glucagon | − | − | − | − | − | − | − |

Candidate markers were not recognized by any antisera with one exception: RBM5, which is a peptide derived from the 5→3 reading of the cDNA encoding angiotensin II, displayed cross-reactivity with antisera against angiotensin II itself. RBM4, however, which was also derived from the angiotensin II complementary DNA, but in the 3→5 direction, did not cross-react with angiotensin II or any other antiserum tested. The fact that all of the other antisera have failed to react with any other biomarker suggests that the criteria employed in their design is generally sound.

The cross-reactivity among the candidate markers is set forth in Tables 3 and 4.

TABLE 3
ELISA Results for Vaccination Marker Activity

| Marker/ | Candidate Marker Antibody Reactivity | | | | | |
|---|---|---|---|---|---|---|
| Combination | RBM1 | RBM2 | RBM4 | RBM5 | RBM6 | RBM7 |
| RBM2, RBM4, | .005 | .031 | .084 | .040 | .039 | −.003 |
| RBM5 | | | | | | |
| RBM4, RBM5 | .000 | .006 | .239 | .050 | −.003 | .003 |
| RBM2, RBM5 | .009 | .027 | −.123 | .101 | .004 | .011 |
| RBM2 | .034 | .219 | −.037 | .041 | −.006 | .002 |
| RBM4 | −.009 | .020 | .079 | .003 | −.035 | −.030 |
| RBM5 | −.018 | .068 | −.231 | .101 | .006 | −.058 |

TABLE 4
Results of ELISA Study of Vaccination Markers Showing
Relative Absorbance
±SE at 405 nm (N = 3)

| | Candidate Marker | | | |
|---|---|---|---|---|
| Marker | RBM1 | RBM4 | RBM5 | RBM6 |
| RBM1 | .55 ± .15** | −.11 ± .14 | .11 ± .05 | .05 ± .07 |
| RBM4 | .21 ± .06 | 2.26 ± .40** | .24 ± .23 | .32 ± .07 |
| RBM5 | .57 ± .25 | .15 ± .07 | 1.5 ± .44** | .50 ± .02 |
| RBM6 | .12 ± .03 | .02 ± .04 | −07 ± .15 | .84 ± .13** |

**P < .01

The data set forth in Table 3 demonstrates that some of the biomarkers cross-react. RMB2 and RBM5 cross-react weakly, but consistently, and a combination of RBM2, RBM4 and RBM5 elicited some responses to RBM6 although no other marker combination or marker by itself elicited this cross-reactivity. Eliminating the use of RBM2 resulted in much cleaner differentiation between the antibody responses elicited by the remaining markers, as set forth in Table 4.

### EXAMPLE 8

Rat inoculations were carried out as described in Example 7, using candidate markers set forth in Table 5. (Candidate markers are comprised as set forth in Table 1 above.) Each (emulsion of 1 mg peptide in 0.1 ml sterile saline plus 0.1 ml Freund's incomplete adjuvant) but using free marker peptides (i.e., not conjugated to KLH). As set forth in Table 5, RBM1 failed to produce measurable immunity, as did RB6 in this set of experiments. RBM2 and RBM7 were both sufficiently antigenic to produce a measurable and consistent antibody response. Furthermore, RBM2 and RBM7 did not interfere with each other's immune responses nor cross-react with other marker candidates.

TABLE 5
ELISA Results for Rats Inoculated with Candidate Markers

| | Candidate Marker Antibody Reactivity | | | | | | |
|---|---|---|---|---|---|---|---|
| Marker/Combination | RBM1 | RBM2 | RBM3 | RBM4 | RBM5 | RBM6 | RBM7 |
| RBM1 | .00 | .20 | −.05 | −.04 | .1 | −.10 | .14 |
| RBM2 | −.02 | 3.34 | −.07 | −.01 | .05 | −.23 | −.05 |
| RBM6 | −.07 | .39 | .02 | −.06 | .19 | −.01 | .12 |
| RBM7 | −.10 | .22 | −.06 | −.12 | .06 | −.10 | .63 |
| RBM1, RBM | .06 | 1.16 | .07 | .02 | .25 | .06 | .06 |
| RBM1, RBM6, RBM7 | .12 | −.03 | .14 | .40 | .24 | .45 | .56 |
| RBM2, RBM6, RBM7 | .21 | 1.89 | .02 | −.07 | −.26 | .34 | .53 |
| CONTROL* | −.01 | .21 | .05 | .03 | .05 | .03 | .05 |

*Combined scores for two umnoculated control rats

### EXAMPLE 9

Four samples of human sera were obtained with appropriate consent. 20 The frozen samples of cell-free sera were thawed and diluted to 1/1000 in phosphate buffered saline at pH 7.0. Aliquots (100 ul) of these diluted antisera were added to ELISA plates pre-adsorbed with the biomarkers as described in Table 1. A goat anti-human-HRP secondary antibody was used to quantitate the amount of antibody bound to the various marker peptides. Non-specific binding (determined by plating antisera onto PVA coated wells) was subtracted from the readings of the individual marker wells. Results are set forth in Table 6.

TABLE 6
Cross-Reactivity of Candidate Markers with Human Sera

| Se- | | | | | | | |
|---|---|---|---|---|---|---|---|
| rum | | | | | | | |
| Sam- | Candidate Marker Antibody Reactivity | | | | | | |
| ple | RBM1 | RBM2 | RBM3 | RBM4 | RBM5 | RBM6 | RBM7 |
| 1 | .16 | .26 | .07 | .08 | .22 | .22 | −.11 |
| 2 | .01 | .05 | .05 | .20 | .21 | .24 | .00 |
| 3 | .08 | .27 | −.07 | −.16 | −.02 | −.09 | .04 |
| 4 | .01 | −.01 | −.01 | −.02 | −.01 | −.03 | −.02 |

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

1.-177. (canceled)

178. A biomarker composition for administration to a human or other animal subject, comprising:
(a) an antigenic marker; and
(b) a pharmaceutically-acceptable carrier;
wherein said marker does not elicit a vaccination immune response in said subject.

179. A biomarker composition according to claim 178, wherein said antigenic marker is a peptide or peptoid.

180. A biomarker composition according to claim 179, wherein said marker has from about 8 to about 100 amino acid residues.

181. A biomarker composition according to claim 180, wherein said marker has from about 12 to about 30 amino acid residues.

182. A biomarker composition according to claim 179, wherein said marker is a peptide consisting essentially of common amino acids.

183. A biomarker composition according to claim 182, wherein said marker comprises at least one amino acid selected from the group consisting of His, Trp, Arg, Met, Gln, Tyr, and mixtures thereof.

184. A biomarker composition according to claim 183, wherein at least about 50% of the amino acids in said marker are selected from said group.

185. A biomarker composition according to claim 178, further comprising a second antigenic marker, wherein said second antigenic marker does not raise a vaccination immune response in said subject.

186. A biomarker composition according to claim 178, which is an injectable dosage form.

187. A biomarker composition according to claim 178, wherein said composition does not contain a vaccination antigen.

188. A biomarker composition according to claim 178, additionally comprising a vaccination antigen.

189. A biomarker composition according to claim 188, wherein said vaccination antigen creates an immune response in said subject that is substantially indistinguishable from the immune response created by the organism against which the vaccination is effective.

190. A biomarker composition according to claim 188, wherein said vaccination antigen is selected from the group consisting of antituberculosis vaccine, human immunodeficiency virus vaccine, hepatitis C vaccine, Epstein-Barr virus vaccine, cytomegalovirus virus vaccine, E. coli vaccine, feline leukemia virus vaccine, foot-and-mouth virus vaccine, and canine distemper vaccine.

191. A method of raising unique antibodies in an animal subject, comprising administering to said subject a composition comprising an antigenic marker, wherein said marker does not elicit a vaccination immune response in said subject.

192. A method according to claim 191, wherein said marker has from about 16 to about 24 amino acid residues.

193. A method according to claim 191, wherein said marker is a peptide comprising at least one amino acid selected from the group consisting of His, Trp, Arg, Met, Gln, Tyr, Cys, Phe, Asn, Asp, Lys, and mixtures thereof; wherein at least about 50% of the amino acids in said marker are selected from said group.

194. A method according to claim 191, further comprising a second antigenic marker, wherein said second antigenic marker does not raise a vaccination immune response in said subject.

195. A method according to claim 191, additionally comprising a vaccination antigen.

196. A method of identifying an animal subject, comprising performing the method of claim 191.

197. A method according to claim 196, wherein said subject is an agricultural livestock animal.

198. A method according to claim 196, wherein said subject is a domestic companion animal.

199. A method of immunizing an individual at risk of being infected by a pathogen, comprising administering to said individual a vaccine comprising:
(a) a vaccination antigen; and
(b) an antigenic marker;
wherein said antigenic biomarker elicits an antibody in said individual that is different from antibodies elicited by said vaccination antigen.

200. A method according to claim 199, wherein said marker has from about 16 to about 24 amino acid residues, at least 50% of which are selected from the group consisting of His, Trp, Arg, Met, Gln, Tyr, Cys, Phe, Asn, Asp, Lys, and mixtures thereof.

201. A method according to claim 199, wherein said vaccination antigen is selected from the group consisting of antituberculosis vaccine, human immunodeficiency virus vaccine, hepatitis C vaccine, Epstein-Barr virus vaccine, cytomegalovirus virus vaccine, E. Coli vaccine, feline leukemia virus vaccine, foot-and-mouth virus vaccine, and canine distemper vaccine.

202. A method for marking an individual subject having a given characteristic in a population of animal subjects, comprising:
(a) identifying an individual subject having said characteristic; and
(b) administering to said individual a marker composition comprising an antigenic marker that does not elicit a vaccination immune response in said subject.

203. A method according to claim 202, wherein said antigenic marker has from about 16 to about 24 amino acid residues, at least 50% of which are selected from the group consisting of His, Trp, Arg, Met, Gln, Tyr, Cys, Phe, Asn, Asp, Lys, and mixtures thereof.

204. A method according to claim 202, wherein said marker composition additionally comprises a second antigenic marker, wherein said second antigenic marker does not elicit a vaccination immune response in said subject.

205. A method according to claim 202, wherein said population comprises agricultural livestock animals.

206. A method according to claim 202, wherein said population comprises domestic companion animals.

207. A method for identifying subjects having a given characteristic in a population of animal subjects, comprising:
(a) administering to individual subjects having said characteristic a marker composition comprising an antigenic marker that does not raise a vaccination immune response in said subject; and
(b) screening said population to identify individuals having said characteristic by the presence of an antibody to said marker in said individual.

208. A method according to claim 207, wherein said antigenic marker is a peptide having from about 8 to about 100 amino acid residues, consisting essentially of common amino acids.

209. A method according to claim 208, wherein at least 50% of said amino acid residues are selected from the group consisting of His, Trp, Arg, Met, Gin, Tyr, and mixtures thereof.

210. A method according to claim 207, wherein said marker composition additionally comprises a second antigenic marker, wherein said second antigenic marker does not elicit a vaccination immune response in said subject.

211. A method according to claim 207, wherein said population comprises agricultural livestock animals.

212. A method according to claim 207, wherein said population comprises domestic companion animals.

213. A method according to claim 207, wherein said characteristic is the vaccination of said subject against a given disease.

214. An identification kit, for identifying an animal subject having been administered an antigenic marker, comprising:
(a) said antigenic marker or antigenic equivalent thereof; and
(b) an indicator which provides a signal when a tissue sample from said subject is contacted with said marker.

215. An identification kit according to claim 214, wherein said antigenic marker is a peptide or peptoid having from about 8 to about 100 amino acid residues.

216. An identification kit according to claim 215, wherein said marker comprises at least one amino acid selected from the group consisting of His, Trp, Arg, Met, Gln, Tyr, Cys, Phe, Asn, Asp, Lys, and mixtures thereof.

217. An identification kit according to claim 214, wherein said indicator is an enzyme linked immunosorption assay.

218. An antigenic marker peptide, comprising from about 12 to about 30 amino acid residues at least about 50% of which are selected from the group consisting of His, Trp, Arg, Met, Gln, Tyr, Cys, Phe, Asn, Asp, Lys, and mixtures thereof, wherein said marker is substantially immunogenic, antigenically unique, and does not create a significant adverse immune response in a human or other animal subject.

219. A biomarker composition according to claim 218, wherein said marker has from about 16 to about 24 amino acid residues.

220. A biomarker composition according to claim 218 having an average attribute score of less than about 11.

221. A method of selecting a marker, comprising:
(a) identifying a candidate marker having from about 8 to about 100 amino acid residues;
(b) determining the attribute score for each of said residues in said candidate marker, said attribute score being the average of the rarity, antigenicity and substitutability scores, on a scale of from 1 to 20, for each of said residues;
(c) calculating the average attribute score for all of said residues in said candidate marker; and
(d) selecting said candidate marker if said average attribute score is less than about 11.

222. A method according to claim 221, further comprising screening said candidate marker against a database of naturally occurring peptide sequences and rejecting said candidate marker if said candidate marker is homologous to a peptide sequence in said database.

223. A method according to claim 222, wherein said candidate marker is rejected if said candidate marker is more than about 20% homologous with said peptide sequence.

224. A method according to claim 222, wherein said candidate marker is rejected if it shares more than one triplet region with a protein in said database.

225. A marker selected by a method of claim 221.

226. A tracking system for tracking individual animal subjects having a characteristic in a population of subjects, comprising:
(a) a marker composition comprising an antigenic marker that does not elicit a vaccination immune response in said subject;
(b) a marking system, for administering said marker composition to said subjects having said characteristics; and
(c) a testing system, for screening subjects in said population to identify said subjects who have been administered said antigenic marker.

227. A tracking system according to claim 226, wherein said antigenic marker is a peptide or peptoid having from about 8 to about 100 amino acid residues.

228. A tracking system according to claim 226, additionally comprising a database for storing information regarding subjects to whom said marker composition has been administered.

229. A tracking system according to claim 227, wherein said characteristic is the status of having been vaccinated against a given pathogen.

230. An administration system for tracking animal subjects in a population of subjects having characteristics comprising a first characteristic and a second characteristic, wherein:
(a) said system associates a first antigenic marker with said first characteristic;
(b) said system associates a second antigenic marker with said second characteristic, wherein said second antigenic marker is antigenically unique relative to said first antigenic marker; and
(c) said antigenic markers do not elicit a vaccination response in said subjects.

231. An administration system according to claim 230, wherein said first characteristic is unrelated to said second characteristic.

232. A method for tracking individual animal subjects having a characteristic in a population of subjects having a plurality of characteristics, comprising:
(a) providing an marker composition which is associated with said characteristic, wherein said composition comprises an antigenic marker that does not raise a vaccination immune response in said subjects;
(b) administering said marker composition to subjects having said characteristic; and
(c) screening subjects in said population to identify subject who have been administered said marker composition.

233. A method according to claim 232, for tracking subjects having a first characteristic and subjects having a second characteristic, wherein said providing step comprises associating a first antigenic marker with said first characteristic; and associating a second antigenic marker with said second characteristic, wherein said second antigenic marker is antigenically unique relative to said first antigenic marker.

234. A method according to claim 233, wherein said first characteristic is the status of having been immunized against a first pathogen and said second characteristic is the status of having been immunized against a second pathogen.

235. A database for use in a system for tracking animal subjects having a plurality of characteristics, comprising:
(a) the composition of a marker composition comprising an antigenic marker, wherein said antigenic marker does not elicit a vaccination immune response in said subjects; and
(b) identification of a characteristic with which said marker is associated.

236. A database according to claim 235, wherein said antigenic markers are peptides or peptoids having from about 8 to about 100 amino acid residues, at least 50% of which are selected from the group consisting of His, Trp, Arg, Met, Gin, Tyr, Cys, Phe, Asn, Asp, Lys, and mixtures thereof.

237. A biomarker composition, for administration to a human or other animal subject, comprising:
(a) a plurality of antigenic markers; and
(b) a pharmaceutically-acceptable carrier.

238. A biomarker composition according to claim 237, comprising from 4 to 8 antigenic markers wherein said antigenic markers or peptides or peptoids comprising from about 12 to about 30 amino acid residues.

239. A method for marking an individual subject having a given characteristic in a population of animal subjects, comprising:
(a) identifying an individual subject having said characteristic; and
(b) administering to said individual a plurality of antigenic markers.


SEQUENCE LISTING (20 sequences)

SEQ ID NO: 1 - protein, 18 aa, Artificial Sequence
  Synthetically derived polypeptide
HWRWRMRMHWHMWRQRMW

SEQ ID NO: 2 - protein, 18 aa, Artificial Sequence
  Synthetically derived polypeptide
HWHWHWHWHWHWHWHWHW

SEQ ID NO: 3 - protein, 18 aa, Artificial Sequence
  Synthetically derived polypeptide
HWRHWMHWQHWYHWCHWF

SEQ ID NO: 4 - protein, 16 aa, Artificial Sequence
  Synthetically derived polypeptide
YHMWYHMWYHMWYHMW

SEQ ID NO: 5 - protein, 12 aa, Artificial Sequence
  Synthetically derived polypeptide
WCMHTFWCMHTF

SEQ ID NO: 6 - protein, 12 aa, Artificial Sequence
  Synthetically derived polypeptide
WPHDMCWFHDMC

SEQ ID NO: 7 - protein, 20 aa, Artificial Sequence
  Synthetically derived polypeptide
RRYFMYRRYFMYRRYFMYRR

SEQ ID NO: 8 - protein, 12 aa, Artificial Sequence
  Synthetically derived polypeptide
YFMYRRYFMYRR

SEQ ID NO: 9 - protein, 16 aa, Artificial Sequence
  Synthetically derived polypeptide
HWRWRMRMHWRWRMRM

SEQ ID NO: 10 - protein, 16 aa, Artificial Sequence
  Synthetically derived polypeptide
DWFHDWFHDWFHDWFH

SEQ ID NO: 11 - protein, 11 aa, Artificial Sequence
  Synthetically derived polypeptide
YFMYRRYFMRR

SEQ ID NO: 12 - protein, 8 aa, Artificial Sequence
  Synthetically derived polypeptide
HWRWRMRM

SEQ ID NO: 13 - protein, 8 aa, Artificial Sequence
  Synthetically derived polypeptide
WPHDMCWP

SEQ ID NO: 14 - protein, 10 aa, Artificial Sequence
  Synthetically derived polypeptide
HRNYFHRNYF

SEQ ID NO: 15 - protein, 14 aa, Artificial Sequence
  Synthetically derived polypeptide
WFYMYMYFWFYMYF

SEQ ID NO: 16 - protein, 10 aa, Artificial Sequence
  Synthetically derived polypeptide
RLAHMYVGKT

SEQ ID NO: 17 - protein, 8 aa, Artificial Sequence
  Synthetically derived polypeptide
EGVYVHPV

SEQ ID NO: 18 - protein, 11 aa, Artificial Sequence
  Synthetically derived polypeptide
ETMKLVTGSPS

SEQ ID NO: 19 - protein, 13 aa, Artificial Sequence
  Synthetically derived polypeptide
EETGVTKTFMTDK

SEQ ID NO: 20 - protein, 12 aa, Artificial Sequence
  Synthetically derived polypeptide
WFYMYFWFYMYF